(19) United States
(12) Patent Application Publication
(10) Pub. No.: US 2021/0338481 A1
(43) Pub. Date: Nov. 4, 2021

(54) Lacrimal gland implant for drug delivery and method

(71) Applicant: Bruce B. Becker, Malibu, CA (US)

(72) Inventor: Bruce B. Becker, Malibu, CA (US)

(21) Appl. No.: 17/235,227

(22) Filed: Apr. 20, 2021

Related U.S. Application Data
(60) Provisional application No. 63/012,611, filed on Apr. 20, 2020.

(51) Int. Cl. A61F 9/00

(52) U.S. Cl. CPC A61F 9/0026

(57) ABSTRACT

An implant placed into the lacrimal gland of a patient for delivery of a drug to the eye for treating conditions such as glaucoma, or merely the gland itself for treating dry eye syndrome. The implant can be placed in such a way that the drug-carrying surfaces of the implant are exposed to the glandular tissues allowing diffusion of the drug into those tissues. For glaucoma, tears generated by those tissues can contain an amount of the drug for delivery to the surface of the eye. The implant can be made from biodegradable/bioabsorbable or non-biodegradable/non-bioabsorbable materials. A tool and method of use facilitates proper emplacement of the implant in part of the lacrimal gland. The tool can include a hallow needle having an axial bore through which the implant passes. During emplacement, a ramrod connected to a physician-manipulable plunger on a syringe-like device pushes the implant from the tip of the needle. Physical structures on the implant can help properly release the drug carried by the implant and help maintain its position in the lacrimal gland.

### PRIOR APPLICATION

This application claims the benefit of U.S. Patent Provisional Application Ser. No. 63/012,611, filed 2020 Apr. 20, incorporated herein by reference.

### FIELD OF THE INVENTION

This invention relates to drug therapy and more particularly to the placement of a drug releasing implant into the lacrimal gland.

### BACKGROUND

Topical medications are used to treat many eye diseases, such as glaucoma, infection, and inflammation. However, non-compliance with the application of topical medications is a significant problem.

Eyedrops must be instilled daily, often many times a day. Patient compliance is often inadequate leading to subtherapeutic dosing. This can lead to loss of vision and in many cases blindness.

This is especially an increasing problem in glaucoma. It is believed that glaucoma affects 2.2 million people in the United States and 67 million people worldwide. It is estimated that the number of glaucoma patients will increase by 50% in the next 15 years. Patients may fail or are unable to instill drops daily or to instill all of their daily drops. Many patients are unable to properly instill drops. Ineffective technique may lead to inadequate dosing or excessive use of drops resulting in side effects or costly waste of drops. In addition the peak effect of many drops is within two hours of being instilled. This leaves an inadequate effect for much of the time requiring frequent application of drops, further reducing compliance. Less than 1 to 5% of topically administered drops reach the aqueous humor. This results in a minimal effect since drops are only administered 1 to 4 times daily, rather than continuously.

Injections of medications are sometimes performed. However, this is painful, requires a doctor visit and has a short-lasting effect.

As a result, drug releasing implants have been proposed for drug therapy as disclosed in Tu, et al., U.S. patent Ser. No. 10/406,029. Such implants are intended to release medication that would flow over the eye, and would release medication over a significant period of time. Some types of implants can be removed when the drug is depleted and a new implant placed. Such implants typically consist of a retention structure and a drug core. Other types of implants are biodegradable and do not need to be removed when the drug has been entirely released. Implants may produce a burst release whereby much of the drug is released early and less is available for later release. Biodegradable implants may have a greater burst release than non-biodegradable implants.

Drug releasing implants may be placed on the surface of the eye or the surface of adjacent structures. Such implants release drug onto the surface of the eye. The amount released and the region of drug release can make the coverage of the eye and penetration into the eye variable. An implant placed in the superior conjunctival cul-de-sac is one such drug releasing implant. However, the implant is not visible to the patient. Therefore the patient may be unaware of an unwanted extrusion of the implant. There may be a period of time when no medication is administered potentially resulting in visual loss. Frequent visits to the doctor can be required to check the implant thus producing the same compliance problem that the implant is supposed to avoid.

An extended wear contact lens that releases drug is being developed as disclosed in Ciolino, et al., U.S. Pat. No. 8,414,912. This extended wear contact lens has to be regularly replaced, such as monthly. This can be difficult for many elderly patients and require monthly doctor visits, raising the same compliance issues noted above. Furthermore, the incidence of corneal ulcers, which can cause severe visual loss, is 10 to 15 times higher with extended wear contact lenses than with daily wear contact lenses.

A drug releasing implant that is placed on the surface of the eye has been proposed for example in Leahy, et al., U.S. Pat. No. 8,167,855. It also has the problem of extrusion unrecognized by the patient. Further, release of the drug may not be uniform over the eye.

A drug releasing implant can be placed in the lacrimal drainage system, specifically the punctum and canaliculus, as disclosed in de Juan, Jr., et al., U.S. Pat. No. 7,998,497. An implant has been developed for the short term release of a corticosteroid. The lacrimal drainage system starts in the very medial upper and lower eyelids and is a series of ducts that drain tears into the nose. There are puncta on the medial upper and lower lids which drain into the upper and lower canaliculi, leading to the common canaliculus, then on into the lacrimal sac and down the nasolacrimal duct into the nose.

A drug releasing implant for placement into the punctum and canaliculus for longer term release of glaucoma medication has been proposed, as disclosed in Rapaki, et al., U.S. patent Ser. No. 10/434,009. The punctum which is the opening of the lacrimal drainage system to the ocular surface is small (0.3 mm). Therefore a very small surface area is available for release of the drug to the ocular surface. Repeated dilation of the punctum to place the implant is painful and makes extrusion more likely each time a new implant is placed. For example, lacrimal punctal plugs for dry eye syndrome have a very high extrusion rate.

Drug releasing implants have also been injected into the eye. Ozurdex brand implant available from Allergan of Dublin, Ireland can be injected into the vitreous gel. It releases dexamethasone. The Ozurdex implant is a biodegradable implant used to treat diseases of the vitreous and retina such as diabetic retinopathy, uveitis, and macular edema.

Non-biodegradable drug releasing implants have been proposed for the vitreous cavity. Vitrasert brand implant available from Bausch & Lomb Incorporated of Irvine, Calif. can be implanted for cytomegalovirus (CMV) retinitis. It releases ganciclovir for five to eight months. Retisert brand implant also available from Bausch & Lomb is a drug releasing implant that releases fluocinolone for 2.5 years for chronic noninfectious uveitis. Iluvien releases fluocinolone in the vitreous cavity. Surodex brand implant available from Oculex Pharmaceuticals of Sunnyvale, Califonia is a metal implant that releases dexamethasone in the vitreous cavity for uveitis.

Subconjunctival injection of liposome loaded latanoprost and subconjunctival injection of microspheres of timolol maleate have been proposed. Drug releasing implants placed subconjunctivally have also been evaluated to treat glaucoma.

The surgical implantation of drug releasing implants in the anterior chamber also is under evaluation to treat glaucoma as disclosed in Tu, et al., U.S. Pat. No. 9,066,782. Such procedures require intraocular surgery and carry a risk of blinding endophthalmitis. Also drug releasing implants in the vitreous gel to treat retinal disease have been proposed, as disclosed for example in Nivaggioli, et al., U.S. Pat. No. 8,685,435.

None of the above described implants appear to release medication diffusely and completely over the ocular surface. Therefore the penetration of medication into the interior is irregular and suboptimal. All appear to have the risks of infection, and extrusion. Some require major intraocular surgery. Many result in an inability of the patient to evaluate their presence.

The lacrimal gland produces tears. As shown in FIG. 1 it is a tubuloacinar structure. There are multiple lobules 3 separated by connective tissue. Each lobule is composed of multiple acini 4. Each acinus is a grape-like cluster of multiple lacrimal glandular cells that surround a central lumen 5 or cavity. The apex 6 of each lacrimal glandular secretory cell 7 points toward the lumen. The lacrimal glandular cells are all serous cells that produce a watery serous fluid. The central lumen of multiple acini converge to form intralobular ducts 8. These intralobular ducts converge to form interlobular ducts 9. The interlobular ducts then converge to form the secretory ducts 10.

As shown in FIG. 2 the lacrimal gland 1 has an orbital lobe 11 and a palpebral lobe 12. The palpebral and orbital lobes are divided by the lateral horn of the levator aponeurosis 13. These two lobes are joined posteriorly. The orbital lobe lies in the superotemporal orbit in the lacrimal fossa posterior to the orbital septum 17. The palpebral lobe of the lacrimal gland lies in the superotemporal conjunctival cul-de-sac. The secretory ducts 14 of the orbital lobe of the lacrimal gland extend from the orbital lobe to pass through the palpebral lobe and empty into the superior lateral conjunctival cul-de-sac 16. The ducts 15 from the palpebral lobe of the lacrimal gland also empty into the superior lateral conjunctival cul de sac. The tears produced by the lacrimal gland continually empty onto the ocular surface and then bathe the entire surface of the eye.

There are also multiple accessory lacrimal glands. The accessory lacrimal glands are only 10% of the volume of the orbital and palpebral lacrimal glands. The accessory lacrimal glands of Krauss and Wolfring are located primarily in the superior conjunctival cul-de-sac and just superior to the tarsus respectively. A much smaller number of accessory lacrimal glands are located in the inferior conjunctival cul-de-sac.

The tear film has three layers. An outer lipid layer produced by the Meibomian glands reduces tear evaporation. The Meibomian glands are present throughout the eyelids. The thickest layer is the central aqueous layer which is produced by the lacrimal gland. The deepest layer is the mucus layer. It is produced by the goblet cells in the conjunctiva. The mucus layer aids in spreading tears over the ocular surface.

It is important to note again that the lacrimal drainage ducts are anatomically and functionally distinct from the lacrimal gland. The lacrimal drainage ducts are located in the extreme medial lids, lacrimal fossa, and nose. The puncta open on the medial upper and lower lids into the upper and lower canaliculi which come together as the common canaliculus which enters the lacrimal sac, then to the nasolacrimal duct, and into the nose. The only purpose of the lacrimal drainage system is to drain tears into the nose.

Therefore, there is a need for a drug delivery device and method which can supply medication to the eye in such a way which addresses one or more of the above problems.

Dry Eye Syndrome

Another common affliction is known as dry eye syndrome. Symptoms include burning, foreign body sensation, irritation, itching, redness, and reduced vision. A reduced production of tears by the lacrimal gland is the most frequent cause of dry eye syndrome. Another cause is evaporative dry eye. Abnormal lipid production can result from inflammation of the meibomian glands of the lids (blepharitis). The abnormal lipid later does not adequately prevent tear evaporation.

Dry eye syndrome is usually idiopathic. Another entity is sjogren's syndrome. Sjogren's syndrome is an autoimmune disorder with dry eyes and dry mouth. Primary Sjogren's syndrome has anti-Sjogren's A or B antibodies. Secondary Sjogren's syndrome is dry eye syndrome and often dry mouth associated with a systemic connective tissue disease such as rheumatoid arthritis or systemic lupus erythematosus. The lacrimal gland of patients with dry eye syndrome exhibit chronic inflammation, fibrosis, and atrophy of the acini. There is also chronic inflammation of the ocular surface from the dryness.

Treatment of dry eye syndrome has often included topical lubricants such as artificial tears or gel during the day and lubricating ointment at bedtime. Topical lubricants are often inadequate except for mild dry eye syndrome. Artificial tears have a brief duration of action. Even frequent application of artificial tears does not reproduce the constant production of tears by the lacrimal gland. Gels have a longer duration but often cause blurring.

Lacriserts are small pellets that dissolve while emitting artificial tears over a 12 hour period. The pellets are placed in the inferior conjunctival cul-de-sac. However, patients find the implants difficult to place and they may extrude. An improvement in symptoms has been minimal. As a result Lacriserts have failed to gain patient or physician acceptance.

Another prior method to treat dry eye syndrome is occlusion of the puncta or canalicluli to prevent or stop tear drainage through the lacrimal drainage system into the nose. An increased layer of tears over the surface of the eye results from occlusion of one or both puncta and in some cases canaliculi. Punctal plugs are often used to occlude the puncta. The plugs are made of silicone or a biodegradable material. The puncta may also be occluded with a procedure using cautery. In spite of punctal occlusion many patients continue to have symptoms from their dry eye syndrome.

There is chronic inflammation of the ocular surface from tear deficiency in dry eye syndrome. Present and future treatments for dry eye syndrome are targeted to reducing inflammation of the ocular surface. Cyclosporin eyedrops (Restasis, Cequa) and lifitigrast (Xiidra) eyedrops have been used for dry eye syndrome. These drops inhibit T cell proliferation and migration and thereby reduce inflammation of the ocular surface. Corticosteroid eyedrops may be used briefly to reduce ocular surface inflammation. However, corticosteroids have multiple side effects such as glaucoma and cataracts and cannot be used chronically. Other products in development appear to target the reduction of ocular surface inflammation, without treatment of tear production.

Parasympathetic innervation of the lacrimal gland stimulates tear production. Systemic pilocarpine, a parasympathetic agonist has been used to increase saliva production for dry mouth and increase tear production. However it has multiple side effects such as abdominal cramps, diarrhea, nausea, diaphoresis and flushing that have greatly limited use. Androgen therapy has been proposed as a treatment for dry eye syndrome. However, eyedrops are poorly absorbed by the lacrimal gland. Systemic androgens have multiple side effects and therefore cannot be used by most patients.

Apparently no current therapy adequately treats dry eye syndrome. Apparently no current or developing therapy targets the lacrimal gland which is the source of decreased tear production.

Therefore, there is a need for an effective treatment of dry eye syndrome which addresses one or more of the above problems.

### SUMMARY

The principal and secondary objects of the invention are to provide improved drug delivery device and method for supplying a drug to the eye, or the improved treatment of dry eye syndrome.

These and other objects are achieved by a drug-releasing implant being implanted into the lacrimal gland.

In some embodiments the implant can be biodegradable or non-biodegradable.

In some embodiments a biodegradable drug releasing implant is loaded within the bore of a needle. In some embodiments the needle is attached to a syringe having a plunger with a small diameter distal end engaging the bore and driving the implant out when the plunger is depressed.

In some embodiments there is provided a method for delivering a drug to an eye or its associated lacrimal system to treat a condition, said method comprises: placing an implant into a lacrimal gland of a patient, wherein the implant comprises: a body comprising: a drug-carrying region comprising: a drug selected to treat said condition; and, wherein said placing comprises: positioning said implant so that an exposed surface of the drug-carrying region is directly exposed to glandular tissues; and, diffusing of said drug into said glandular tissues.

In some embodiments the method further comprises: allowing secretions emanating from said glandular tissues to carry an amount of said drug diffused into said glandular tissues through one or more lacrimal gland secretory ducts, and onto the surface of the eye.

In some embodiments the method further comprises: selecting said drug to treat dry eye syndrome; allowing said drug diffused into said glandular tissues to stimulate enhanced secretory activity; and, increasing secretions emanating from said glandular tissues through a one or more lacrimal gland secretory ducts, and onto the surface of the eye.

In some embodiments said implant consists of one or more biodegradable and/or bioabsorbable materials.

In some embodiments said implant comprises one or more biodegradable and/or bioabsorbable materials.

In some embodiments said implant comprises one or more non-biodegradable and/or non-bioabsorbable materials.

In some embodiments said placing comprises inserting said implant in a palpebral lobe of said lacrimal gland.

In some embodiments said placing comprises inserting said implant in an orbital lobe of said lacrimal gland.

In some embodiments said placing comprises: inserting a first implant in an orbital lobe of said lacrimal gland; and inserting a second implant in a palpebral lobe of said lacrimal gland.

In some embodiments said placing comprises: inserting two or more of said implants into said lacrimal gland.

In some embodiments the method further comprises: removing said implant out of said lacrimal gland; wherein said removing comprises: grasping a part of said implant with a grasping tool.

In some embodiments the method further comprises retaining said implant in the lacrimal gland during delivery of said drug onto said eye.

In some embodiments said retaining occurs in absence of fixating said implant to the lacrimal gland with a clamp or other device apart from said implant.

In some embodiments said retaining comprises providing said implant with at least one radially extended prominence.

In some embodiments said retaining comprises providing said implant with at least one distal anchoring bulge.

In some embodiments said retaining comprises providing said implant with a plurality of spaced apart anchoring bulges.

In some embodiments said retaining comprises providing said implant with a proximal flange.

In some embodiments said implant comprises an integrated combination of said body and said proximal flange; and wherein said drug-carrying region is imbedded in said lacrimal gland while said proximal flange is not.

In some embodiments said proximal flange bears against the conjunctiva of said patient.

In some embodiments said flange has a cross-sectional dimension greater than said drug-carrying region.

In some embodiments said placing comprising inserting said implant through the conjunctiva of said patient.

In some embodiments said placing comprising inserting said implant through an upper eyelid of said patient.

In some embodiments said placing comprises: instilling a topical anesthetic on the surface of the eye; retracting the lateral upper eyelid; instructing the patient to look inferiorly and toward the nose; inserting a needle carrying said implant through the conjunctiva and into the lacrimal gland; ejecting said implant out of said needle into the lacrimal gland; and, withdrawing said needle from said conjunctiva.

In some embodiments said inserting comprises: pushing said needle until a stopper prevents further insertion;

In some embodiments said placing comprises: instilling a topical anesthetic on the surface of the eye; forming an incision in the lateral upper eyelid; creating an opening in the orbital septum; instructing the patient to look inferiorly and toward the nose; inserting a needle carrying said implant through the lateral upper eyelid, orbital septum and into the lacrimal gland; ejecting said implant out of said needle into the lacrimal gland; and, withdrawing said needle from said lateral upper eyelid.

In some embodiments said inserting comprises: pushing said needle until a stopper prevents further insertion.

In some embodiments there is provided a method for treating dry eye syndrome, said method comprises: delivering an effective amount of a drug to the lacrimal gland of a patient; wherein said delivering comprises: placing an implant into a lacrimal gland of a patient, wherein the implant comprises: a body comprising: a drug-carrying region comprising: a drug; and, wherein said placing comprises: positioning said implant so that an exposed surface of the drug-carrying region is directly exposed to glandular tissues; and, diffusing of said drug into said glandular tissues.

In some embodiments there is provided an implant for delivering a drug to an eye or lacrimal system of a patient, said implant comprises: a body comprising: a drug-carrying region comprising: a drug; said implant being shaped and dimensioned to be inserted into the lacrimal gland of the patient; whereby while the implant is inserted into the lacrimal gland of the patient a surface of said drug-carrying region is directly exposed to glandular tissues thereby allowing diffusion of said drug into said glandular tissues.

In some embodiments said body comprises a first biodegradable and/or bioabsorbable material.

In some embodiments said body comprises a first non-biodegradable and/or non-bioabsorbable material.

In some embodiments said implant further comprises at least one radially extended prominence.

In some embodiments said prominence comprises at least one distal anchoring bulge.

In some embodiments said radially extended prominence has a cross-sectional dimension greater than said drug-carrying region.

In some embodiments said implant further comprises a plurality of spaced apart anchoring bulges extending radially from said body.

In some embodiments said implant further comprises a proximal flange.

In some embodiments said drug carrying region is separated a distance from said proximal flange.

In some embodiments said drug carrying region and said flange are made from a unitary, integrated piece of material.

In some embodiments said proximal flange is made from a second biodegradable and/or bioabsorbable material.

In some embodiments said first biodegradable and/or bioabsorbable material and said second biodegradable and/or bioabsorbable material are identical.

In some embodiments said proximal flange comprises a distal flange surface oriented to rest against at least part of the tissue surrounding an opening in said lacrimal gland when said implant is properly emplaced in said lacrimal gland.

In some embodiments said first biodegradable and/or bioabsorbable material is selected from the group consisting of: poly (lactic co glycolic acid) (PLGA); hydroxymethylcellulose; collagen; polydioxanone; E-Caprolactone-L-Lactide-copolymer; polycaprolactone (PCL)/PLGA; mand, polyethylene glycol (PEG).

In some embodiments said body further comprises biodegradable particles selected from the group consisting of: colloidal particles, liposomes, microparticles, nanoparticles and nanospheres

In some embodiments said drug is dispersed in biodegradable polymeric matrix or membrane.

In some embodiments said drug comprises a therapeutic agent selected from the group consisting of: bimatoprost, latanoprost, tafluprost, travoprost, brinzolamide, betaxolol, carteolol, levobunolol, timolol, apraclonidine, brimonidine, ganciclovir, acyclovir, famcyclovir, gentamicin, tobramycin, moxifloxacin, levofloxacin, ocufloxacin, ciprofloxacin, sulfacetamide products, polymyxin, neomycin, penicillin, cephalosporins, doxycycline, tetracycline, minocycline, erythromycin, biaxin, trifluridine, dexamethasone, triamcinolone, fluocinolone, and cyclosporine.

In some embodiments said drug comprises a therapeutic agent selected to treat dry eye syndrome.

In some embodiments said body is coated with a biodegradable material.

In some embodiments said drug is conjugated to a biodegradable material.

In some embodiments said implant establishes a flow of drug-containing fluid of at least 0.01 microliter per minute to said eye.

In some embodiments there is provided the combination of a drug-carrying implant and a tool for inserting said implant into the lacrimal gland; wherein said implant comprises: a drug-carrying region; wherein said tool comprises: a needle having an a sharpened distal tip, an axial bore extending to a distal aperture; a ramrod shaped and dimensioned to slidingly engage said axial bore; wherein said axial bore slidingly carries said implant therein.

In some embodiments said combination further comprises a stopper radially extending from said needle separated an axial distance from said sharpened distal tip.

In some embodiments an axial position of said stopper is adjustable with respect to said distal tip.

In some embodiments said stopper provides a penetration-resisting bearing surface extending beyond a maximum radial extent of said needle.

In some embodiments said penetration-resisting bearing surface extends at least 0.1 mm beyond a maximum radial extent of said needle.

In some embodiments said penetration-resisting bearing surface extends beyond a maximum radial extent of said implant.

In some embodiments said penetration-resisting bearing surface extends at least 0.1 mm beyond a maximum radial extent of said implant.

In some embodiments said penetration-resisting bearing surface is dimensioned to bear directly or indirectly against at least part of the tissue surrounding an opening in said lacrimal gland during insertion of said implant into said lacrimal gland.

In some embodiments there is provided a tool for inserting a drug-releasing implant into the lacrimal gland of a patient, said tool comprises: a hand-graspable member; a needle extending distally from said member; said needle having a distal segment elongated along an axis, said distal segment having a free distal end; and, a stopper located an axial distance proximal from said distal end; wherein said stopper comprises: a radial prominence supporting an penetration-resisting bearing surface a radial distance from said distal segment; wherein said penetration-resisting bearing surface is shaped, dimensioned, and located to directly or indirectly bear against at least part of the tissue surrounding an opening in said lacrimal gland during insertion of said implant into said gland.

In some embodiments said penetration-resisting bearing surface is shaped and dimensioned to prevent over-penetration of said distal segment into said gland.

In some embodiments there is provided a method for emplacing a drug-releasing implant into the lacrimal gland of a patient, wherein said implant has a proximal capping flange having a distal flange surface for resting against the tissue forming said gland, said method comprises: selecting an implantation tool including a needle having a distal segment having a free distal end, and a stopper located an axial distance proximally from said distal end, said stopper supporting a penetration-resisting bearing surface having a radial dimension greater than a maximum radial dimension of said implant; releasably carrying said implant within a bore of said needle; puncturing said gland with said needle to form an opening in the surface of said gland; pushing said needle axially into said lacrimal gland until said penetration-resisting bearing surface prevents further axial movement of said implantation tool; releasing said implant from said implantation tool; and, axially pulling said tool away from said implant.

In some embodiments the penetration-resisting bearing surface directly contacts a portion of the tissue surrounding said opening thereby preventing over-insertion of the needle into the lacrimal gland.

In some embodiments said selecting further comprises: choosing an implantation tool so that said penetration-resisting bearing surface has a radial dimension which is at least 0.1 mm larger than the maximum radial dimension of the needle.

The content of the original claims is incorporated herein by reference as summarizing features in one or more exemplary embodiments.

### BRIEF DESCRIPTION OF THE DRAWINGS

FIG. 1 is a prior art diagrammatic partial cross-sectional view of the anatomical structure of a number of lacrimal gland lobules.

FIG. 2 is a prior art diagrammatic partial cross-sectional side view of the anatomical structure of a human orbit including the lacrimal gland.

FIG. 3 is a diagrammatic partial cross-sectional side view of the anatomical structure of a human orbit including the lacrimal gland and drug-releasing implants inserted therein.

FIG. 4 is a diagrammatic partial cross-sectional view of the anatomical structure of a number of lacrimal gland lobules including a drug-releasing implant inserted therein.

FIG. 5 is a diagrammatic perspective view of a drug-releasing implant for the lacrimal gland according to an exemplary embodiment of the invention.

FIG. 6 is a diagrammatic side view of the drug-releasing implant for the lacrimal gland of FIG. 5.

FIG. 7 is a diagrammatic perspective view of a drug-releasing implant for the lacrimal gland having radial prominences according to an alternate exemplary embodiment of the invention.

FIG. 8 is a diagrammatic side view of the drug-releasing implant for the lacrimal gland of FIG. 7.

FIG. 9 is a diagrammatic perspective view of a drug-releasing implant for the lacrimal gland having liquid permeable body and internal reservoir according to an alternate exemplary embodiment of the invention.

FIG. 10 is a diagrammatic perspective view of a drug-releasing implant for the lacrimal gland having an agglomeration of small particles containing a drug according to an alternate exemplary embodiment of the invention.

FIG. 11 is a diagrammatic side view of the drug-releasing implant for the lacrimal gland including small particles containing a drug carried within an enclosure according to an alternate exemplary embodiment of the invention.

FIG. 12 is a diagrammatic side view of the drug-releasing implant for the lacrimal gland including a proximal capping flange according to an alternate exemplary embodiment of the invention

FIG. 13 is a diagrammatic perspective view of the insertion of a drug-releasing implant into the lacrimal gland using an emplacement tool according to an exemplary embodiment of the invention.

FIG. 14 is a diagrammatic partial cross-sectional side view and an enlargement view of a tool for inserting a drug-releasing implant into the lacrimal gland according to an exemplary embodiment of the invention.

FIG. 15 is a flow diagram of the primary steps for inserting a drug-releasing implant into the lacrimal gland according to an exemplary embodiment of the invention.

FIG. 16 is a diagrammatic perspective view of the insertion of a drug-releasing implant into the orbital lobe of the lacrimal gland according to an exemplary embodiment of the invention.

FIG. 17 is a diagrammatic perspective view of a tool for inserting a drug-releasing implant into the lacrimal gland according to an alternate exemplary embodiment of the invention.

FIG. 18 is a diagrammatic partial cross-sectional side view of a tool for inserting two or more drug-releasing implants into the lacrimal gland according to an exemplary embodiment of the invention.

FIG. 19 is a diagrammatic partial cross-sectional side view of a tool having a depth stopper for inserting a drug-releasing implant into the lacrimal gland according to an alternate exemplary embodiment of the invention.

FIG. 20 is a diagrammatic cross-sectional end view of a tool having a depth stopper including a visualization gap according to an alternate exemplary embodiment of the invention.

FIG. 21 is a diagrammatic partial cross-sectional side view of a tool having an axially adjustable depth stopper for inserting a drug-releasing implant of the type shown in FIG. 12 into the lacrimal gland according to an alternate exemplary embodiment of the invention.

FIG. 22 is a diagrammatic perspective view of the insertion of a drug-releasing implant into the lacrimal gland using an emplacement tool of the type shown in FIG. 19 according to an alternate exemplary embodiment of the invention.

FIG. 23 is a flow diagram of the primary steps for inserting a drug-releasing implant into the lacrimal gland using an emplacement tool of the type shown in FIG. 19 according to an alternate exemplary embodiment of the invention.

FIG. 24 is a diagrammatic perspective view of the insertion of a drug-releasing implant into the orbital lobe of the lacrimal gland using an emplacement tool of the type shown in FIG. 19 according to an alternate exemplary embodiment of the invention.

FIG. 25 is a flow diagram of the primary steps for inserting a drug-releasing implant into the orbital lobe of the lacrimal gland using an emplacement tool of the type shown in FIG. 19 according to an alternate exemplary embodiment of the invention.

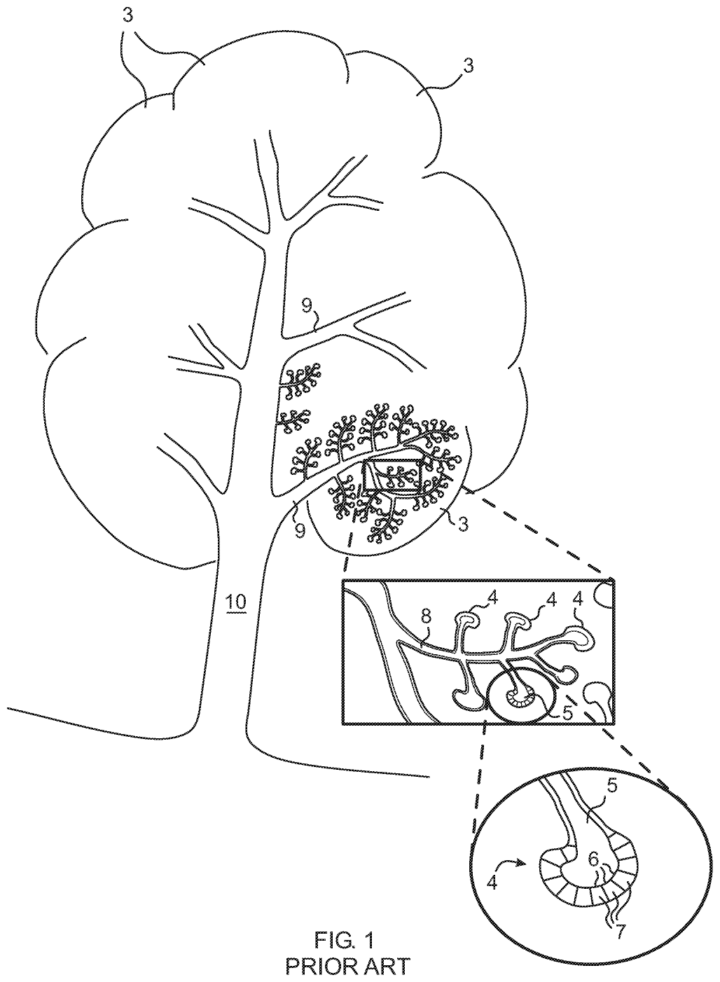

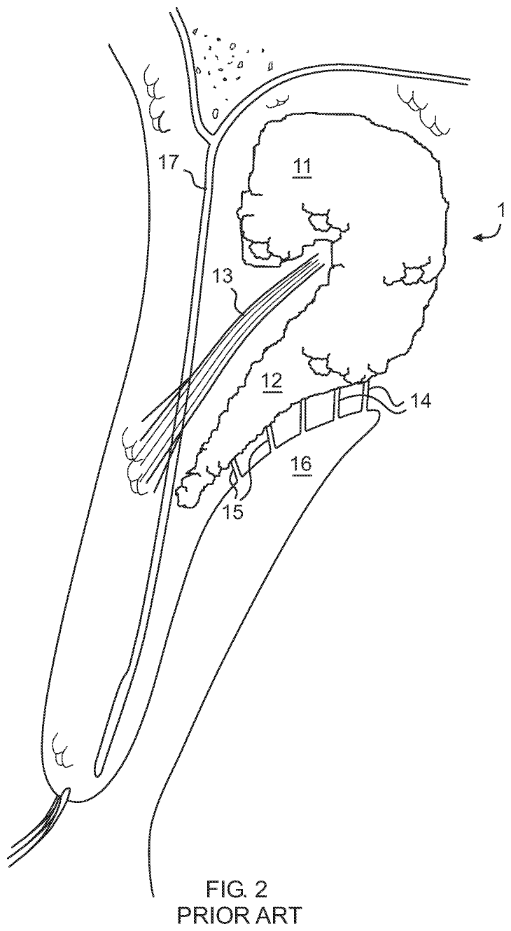

### DESCRIPTION OF THE EXEMPLARY EMBODIMENTS

In this specification, the references to top, bottom, upward, downward, upper, lower, vertical, horizontal, sideways, lateral, back, front, proximal, distal, etc. can be used to provide a clear frame of reference for the various structures with respect to other structures while the patient is upright, and not treated as absolutes when the frame of reference is changed, such as when the device is inverted, disassembled, or the patient is lying down.

If used in this specification, the term “substantially” can be used because manufacturing imprecision and inaccuracies can lead to non-symmetricity and other inexactitudes in the shape, dimensioning and orientation of various structures. Further, use of “substantially” in connection with certain geometrical shapes and orientations, such as “parallel” and “perpendicular”, can be given as a guide to generally describe the function of various structures, and to allow for slight departures from exact mathematical geometrical shapes and orientations, while providing adequately similar function. Those skilled in the art will readily appreciate the degree to which a departure can be made from the mathematically exact geometrical references.

If used in this specification, the word “axial” is meant to refer to directions, movement, or forces acting substantially parallel with or along a respective axis, and not to refer to rotational nor radial nor angular directions, movement or forces, nor torsional forces.

In this specification the units “millimeter” or “millimeters” can be abbreviated “mm”.

In this specification the term “drug” is meant to collectively include one or more medications, therapeutic agents, compounds, chemicals which are to be delivered to the surface of the eye by use of the implant to help treat some condition, or merely delivered to the lacrimal gland itself to treat some condition such as dry eye syndrome.

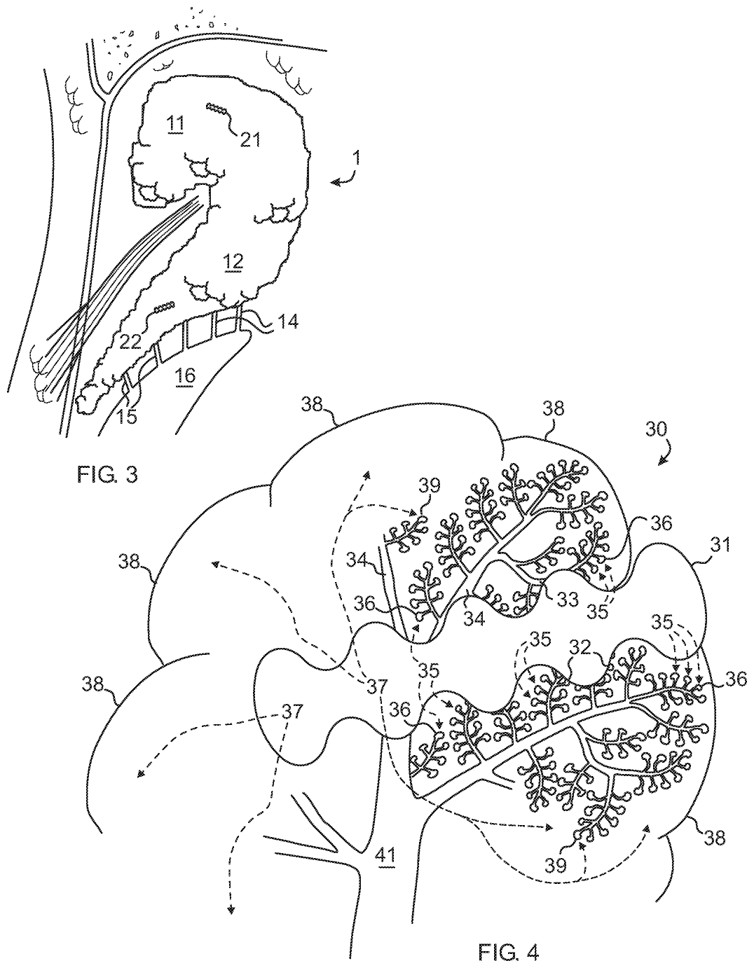

As shown FIG. 3 one or more drug-releasing implants 21,22 can be located in parts of the lacrimal gland 1. For example, one implant 21 can be located in the orbital lobe 11, and another implant 22 can be located in the palpebral lobe 12.

As shown in FIG. 4, a drug-releasing implant 31 can be located in a part of the lacrimal gland 30. Emplacement of the implant, as will be described in greater detail below, is likely to disrupt portions of the structure of the gland including some of the acini 32, intralobular ducts 33, and interlobular ducts 34. However, the acinar cells will continue to generate serous fluid which, in combination with other ambient bodily fluids will flow onto the implant to mix with the drug and lead to rapid diffusion 35 of the drug into the surrounding glandular acini 36. Over time the drug will further diffuse 37 into surrounding lobules 38 and acini 39 within those lobules at a greater distance from the implant.

The tears generated from the acini into which the drug has diffused will now carrying the drug and flow as normal into the network of intralobular, and interlobular ducts and eventually on into the secretory ducts for ultimate delivery to the surface of the eye. Once this flow is established, drug-carrying tears can be continuously supplied to the surface of the eye. The drug which is integrated into the tears completely and constantly coats the surface of the eye resulting in maximal penetration into the eye and maximal clinical effect. It is expected that a properly placed implant in a normally productive lacrimal gland will therefore release between about 0.01 microliter and 1.0 milliliter per minute of drug-carrying tears to the outer surface of the eye.

In other words, the drug carried by the implant is thus released by the implant over a typical designated period of time. The drug diffuses in the lacrimal gland and mixes with the tears that are produced. The tears then exit the lacrimal gland through the lacrimal gland ducts. The release can occur in absence of any intervention by the physician.

It is believed that as the lacrimal gland heals around the emplaced implant, new ductal and connective tissue cells will form to accommodate this established flow of tears over and/or through the implant.

The drug-releasing implant can be made from biodegradable materials which are typically eventually absorbed by the body, in which case the implant need not be removed. In this way, apart from implantation, treatment can occur in absence of further physician intervention until the implant has been degraded. Alternately, the implant can be made from non-biodegradable materials, in which case the implant is eventually removed from the lacrimal gland.

The biodegradable drug-releasing implant for implantation in the lacrimal gland can be made of a variety of materials or their combination. Those materials include biodegradable poly (lactic co glycolic acid) (PLGA), hydroxymethylcellulose, collagen, polydioxanone, E-Caprolactone-L-Lactide-copolymer, polycaprolactone (PCL)/PLGA, polyethylene glycol (PEG), and other biodegradable and/or bioabsorbable materials known to be compatible with the body.

Further, the biodegradable drug-releasing implant can be coated with PLGA, hydroxymethylcellulose, polycaprolactone (PCL)/PLGA and combinations thereof.

Further, the drug can be conjugated to PLGA, hydroxymethylcellulose, PCL/PGLA or combinations thereof.

The biodegradable drug-releasing implant can include or consist of biodegradable colloidal particles, liposomes, microparticles, nanoparticles and nanospheres which carry the drug. In such implants the particles or drug can be dispersed in a biodegradable polymeric matrix or membrane. The biodegradable particles containing the drug be composed of PLGA, PLA, other biodegradable materials, or combinations thereof.

Alternately, nanoparticles containing the drug may be incorporated into biodegradable material such as polyethylene glycol (PEG) or PEG coated nanoparticles.

The non-biodegradable drug-releasing implant for implantation in the lacrimal gland can be made from a variety of materials or their combination. Those materials can include silicone, silicone combined with other polymers, cross-linked hydrogels, NN-dimethylacrylamide, methacrylic acid, poly(ethylene-co-vinyl acetate), microspheres, liposomes, polyvinyl alcohol (PVA), ethylene vinyl acetate, metallic non-ferrous materials and other materials known to be compatible with the body at least for a limited time.

The drug-releasing implant, whether biodegradable, bioabsorbable, non-biodegradable, or non-bioabsorbable can include a number of medications, therapeutics agents, compounds, or chemicals, either alone or in combination for delivery to the eye. Any of the above are referred to as a drug in this specification.

For example, the drug can include one or more glaucoma medications such as bimatoprost, latanoprost, tafluprost, travoprost, brinzolamide, betaxolol, carteolol, levobunolol, timolol, apraclonidine, and brimonidine.

The drug can include one or more anti-infective medications such as ganciclovir, acyclovir, famcyclovir, gentamicin, tobramycin, moxifloxacin, levofloxacin, ocufloxacin, ciprofloxacin, sulfacetamide products, polymyxin, neomycin, penicillin, cephalosporins, doxycycline, tetracycline, minocycline, erythromycin, biaxin, and trifluridine.

The drug can include one or more anti-inflammatory medications such as dexamethasone, triamcinolone, fluocinolone, and cyclosporine.

The drug can include one or more lubricants, artificial tears, and gels.

The drug-releasing implant can be of a variety of shapes.

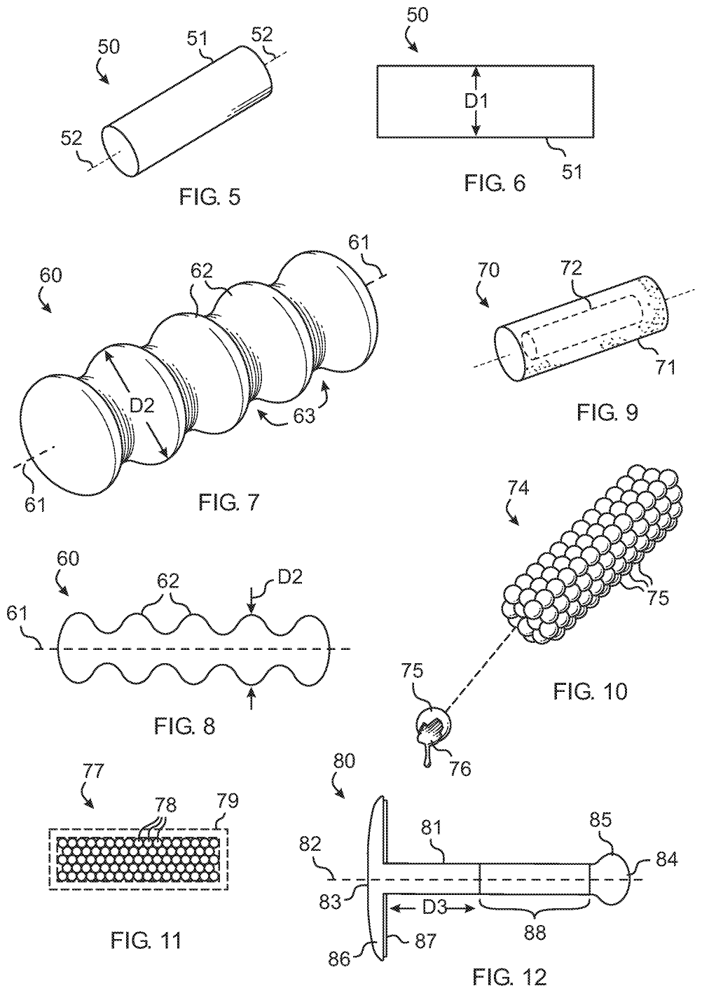

As shown in FIGS. 5-6 the implant 50 can have a substantially cylindrical body 51 having a central axis 52. The body can be elongated along its central axis to form a substantially rod-shaped structure. The body preferably has a relatively small largest outside dimension taken substantially perpendicular to the elongation axis. For a body that is substantially symmetric rotationally, such as a cylinder, that dimension would be a diameter D1 selected to allow the implant to be completely lodged within the lacrimal gland. The relatively small diameter allows easy insertion into the lacrimal gland, and if necessary removal from the lacrimal gland. For implants made of resilient material the dimensions are given for the implant at rest in the lacrimal gland.

FIGS. 7-8 show that other shapes having different diameters on different segments of the body can be used depending on manufacturing, drug impregnation or other concerns. For example, the implant can have a substantially cylindrical body 60 having a central axis 61 and include ridges or other surface features such as one or more radial prominences 62 which increase the surface area of the implant to allow greater exposure to the surrounding glandular structures, and to help prevent axial migration of the implant after emplacement, and create surfaces that increase friction to axial movement. In this embodiment the prominences can be formed by spaced apart bulges separated by grooves 63. The bulges transition to the grooves smoothly to avoid nooks or other structures which would tend to trap debris which could lead to unwanted bacterial propagation. The body preferably has a relatively small largest outside diameter D2 so that it can be completely lodged within the lacrimal gland. In this way the implant can be emplaced in absence of fixating the implant to the lacrimal gland with a clamp or other structure apart from the implant itself.

As shown in FIG. 9 the implant body 70 can be formed as a liquid permeable pellet 71 enclosing drug-carrying reservoir 72.

As shown in FIG. 10 the implant body 74 can be an agglomeration of small particles 75 each containing an amount of drug 76 which can be dispensed into the tissues of the lacrimal gland as the particle coatings dissolve or are otherwise are broken. The particles can be agglomerated to one another by an amount of interconnecting biocompatible glue of a type known in the art.

As shown in FIG. 11 the implant body 77 can be a grouping of small particles 78 each containing an amount of drug which can be dispensed into the tissues of the lacrimal gland as the particle coatings dissolve. The particles can be carried within a biocompatible, liquid permeable enclosure 79.

As shown in FIG. 12 a non-biodegradable implant 80 can have physical features which allow the implant to be easily located and removed after it has ended its drug delivery mission. One such physical feature can be a proximal flange 86 which caps the proximal end of the implant and which remains exposed outside of the lacrimal gland after the implant has been properly emplaced. For example, the implant can include a substantially oblong, substantially cylindrical body 81 extending along an axis 82 between a proximal end 83 and a distal end 84. The distal end can have an anchor 85 formed by a gradual widening of the diameter of the body thereby forming a smooth transition to avoid trapping debris. The proximal end can be capped by the substantially flattened, substantially circular proximal flange 86. The flange can help prevent the implant from being inserted deeper into the lacrimal gland than desired. In some embodiments the proximal flange of the implant can rest on the external surface of the lacrimal gland and in the case of the implant being placed in the palpebral lobe, the proximal flange can rest on the conjunctiva. The remainder of the implant extends into the lacrimal gland so that a drug-carrying region 88 is positioned to be exposed to the internal tissues of the lacrimal gland to permit diffusion of the drug into one or more lobules near the implant. The drug-carrying region can be separated a distance D3 from the flange to allow the drug-carrying region to be more deeply placed. An optional layer of biocompatible adhesive 87 can be present on the distal surface of the flange that faces the lacrimal gland and conjunctiva in order to help prevent extrusion. The flange can also act as a readily exposed grasping point for removing the implant.

Emplacement of the implant will typically depend on whether the implant is to be later removed or to remain in place until it degrades completely.

For a biodegradable or bioabsorbable implant which is intended to remain in the body until it is completely absorbed, the following steps can be undertaken.

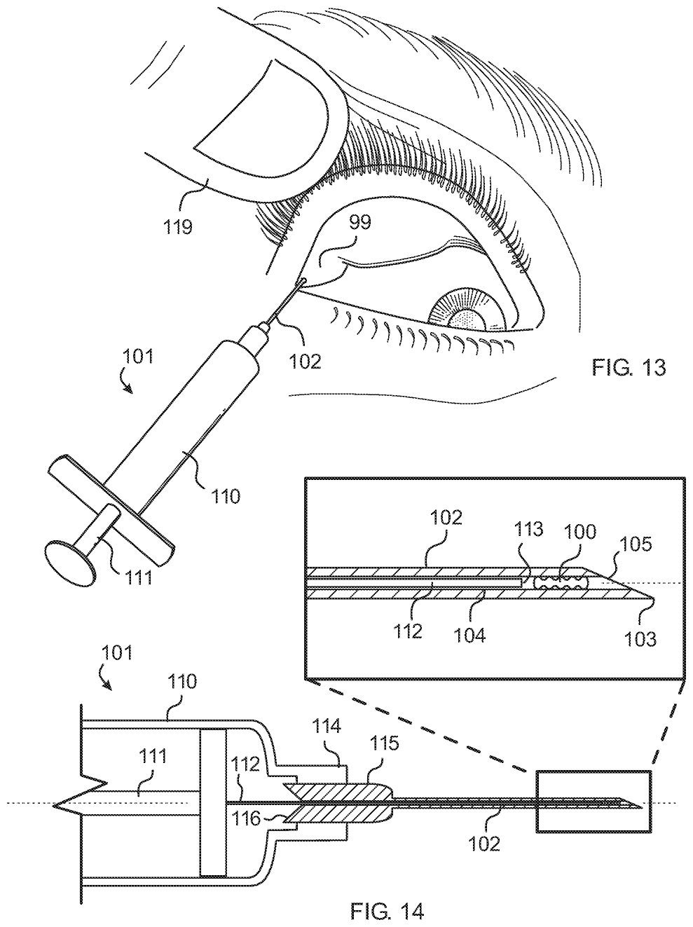

As shown in FIGS. 13-14, the biodegradable drug-releasing implant 100 can be emplaced in the lacrimal gland 99 using an emplacement tool 101. The tool can include a needle 102 having an a sharpened distal tip 103, an axial bore 104 extending to a distal aperture 105. The axial bore is dimensioned to slidingly carry the implant therein. The diameter of the bore can be selected to provide contact with the implant so that a slight amount of static friction exists between the implant and the needle. That friction can be overcome as the implant is being emplaced.

The needle 102 can be attached to the distal end 114 of a syringe-like applicator 110 by a proximal bushing 115. The applicator can include a hand-manipulable plunger 111 attached to a ramrod 112 shaped and dimensioned to slidingly engage the axial bore 104 and have a distal face 113 for bearing against and driving the implant distally in order to eject the implant from the axial bore and out the distal aperture 105. A proximal substantially conical inlet 116 on the bushing can guide the distal face of the ramrod into the axial bore 104 of the needle during assembly.

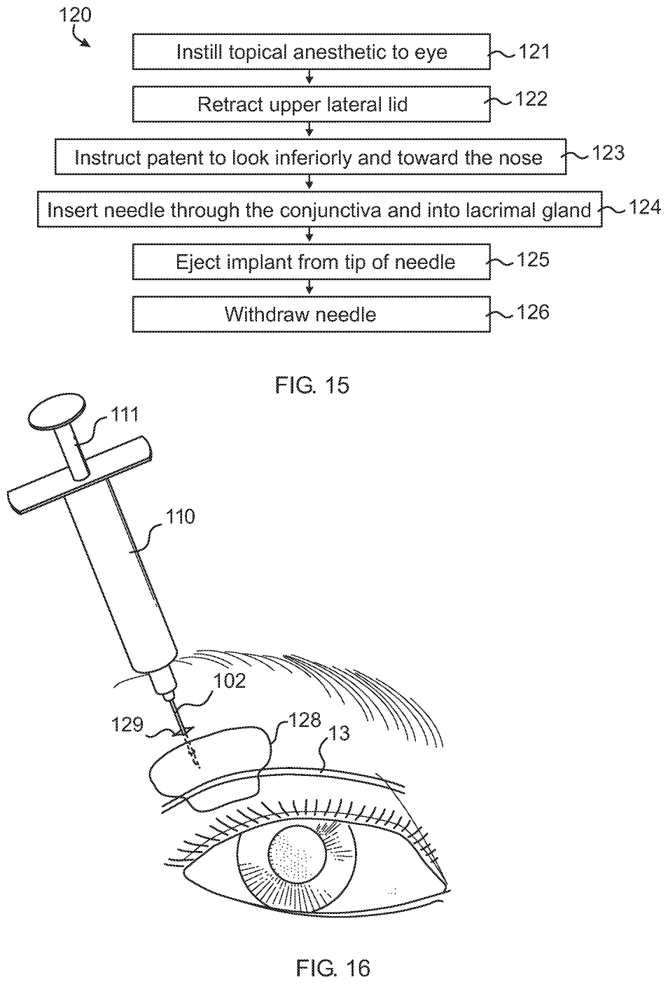

In reference to FIGS. 13 and 15, a method 120 for emplacing the biodegradable implant in the lacrimal gland of a patient is as follows. The physician instills 121 a topical anesthetic on the surface of the eye. The physician then retracts 122 the lateral upper lid with his finger 119 or a retractor. The patient is instructed 123 to look inferiorly and toward the nose. The physician inserts 124 the needle 102 through the conjunctiva and into the lacrimal gland. The physician then depresses the plunger 111 which pushes the ramrod 112 forward to eject 125 the drug-releasing implant 100 into the lacrimal gland. The needle is then withdrawn 126. For some implantations it may be preferable for the physician to push the plunger and eject the implant into the lacrimal gland while the needle is being withdrawn. In this way the implant is placed in the tract created by the needle. In some cases two or more implants can be loaded in the applicator. Both implants can be placed into the lacrimal gland either in the same spot, or in an axially aligned manner while simultaneously withdrawing the needle. Alternately, the first implant can be emplaced in one spot, ad the needle reoriented to another spot to place the second implant.

Alternately, as shown in FIG. 16 the drug-releasing implant can be placed in the orbital lobe 128 of the lacrimal gland. A small incision 129 can be made in lateral upper lid. Blunt opening or sharp opening of the orbital septum is performed. The orbital lobe of the lacrimal gland is identified. The physician places the needle 102 of the applicator 110 into the orbital lobe of the lacrimal gland. The physician then pushes the plunger 111 which ejects the implant into the lacrimal gland. The needle is then withdrawn. In some cases the plunger pushes the implant into the lacrimal gland simultaneously while the needle is withdrawn. This places the implant in the tract created by the needle. In some cases two or more implants can be loaded in the applicator. Both implants can be placed into the lacrimal gland either in the same spot, or in an axially aligned manner while simultaneously withdrawing the needle. Alternately, the first implant can be emplaced in one spot, ad the needle reoriented to another spot to place the second implant.

Alternately, no incision is made. The sharply tipped needle is pushed through the lateral upper lid, orbital septum and into the lacrimal gland. The physician then pushes the plunger which ejects the drug-releasing implant into the lacrimal gland. The needle is then withdrawn. In some cases the plunger pushes the implant into the lacrimal gland simultaneously while the needle is being withdrawn. This places the implant in the tract created by the needle. In some cases two or more implants can be loaded in the applicator. Both implants can be placed into the lacrimal gland either in the same spot, or in an axially aligned manner while simultaneously withdrawing the needle. Alternately, the first implant can be emplaced in one spot, ad the needle reoriented to another spot to place the second implant.

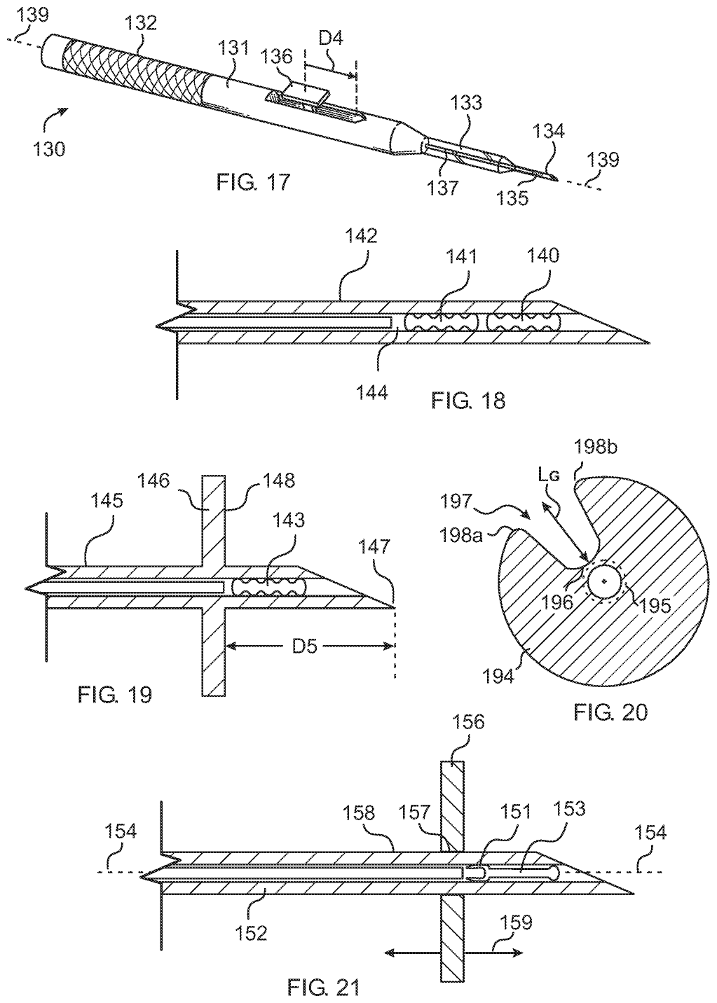

FIG. 17 shows an alternate embodiment of a lacrimal gland implantation tool 130 which can include a hand-manipulable body 131 elongated along an axis 139. The body can have a knurled proximal handle portion 132 and a distal shaft 133 which can terminate in a distal needle 134 carrying a drug-carrying lacrimal gland implant 135. The implant can be ejected by axially sliding a button 136 on the body distally a distance D4 causing a ramrod 137 slidingly engaging the bore of the needle to push the implant out the distal tip of the needle. The shaft can optionally be made of a translucent material to provide the physician greater visibility during use

Alternately, as shown in FIG. 18, two or more implants 140,141 can be loaded adjacently within the bore 144 needle 142 in an axially aligned manner. Both implants can placed into the lacrimal gland either in the same spot or on different spots in the same tract created by the needle by simultaneously withdrawing the needle and ejecting the implants. The implants can be placed in different spots by providing a plunger having markings on the shaft which indicate how far the plunger has been depressed and thereby which of the implants have exited the needle.

As shown in FIG. 19, in order to limit the depth of the location in lacrimal gland that the implant 143 can be emplaced, the needle 145 can be fitted with a stopper 146 formed by a radially expanded skirt located a distance D5 from the sharpened distal tip 147 of the needle. The distal surface 148 of the stopper bears against the tissue surrounding path of the tip of the needle and thus prevents further distal axial movement of the needle. Once the physician detects resistance to further penetration of the needle, the implant can be ejected, and the needle withdrawn. The stopper can be flat and extend 360 degrees around the needle.

Alternately, as shown in FIG. 20, the stopper 194 can extend less than 360 degrees around the needle, thereby creating a gap 197 in the stopper through which the physician can better visualize the insertion of the needle without departing from its penetration preventing function. The gap can extend a radial length LG from the periphery of the stopper to a radially proximal terminus 196 near the periphery of the needle 195. The dimensions of the gap can be maximized to give greater visualization or minimized to provide a greater surface area of the penetration-resisting distal surface for contacting the tissues surrounding the needle and preventing over-insertion of the needle. In this embodiment the gap is shown having a generally trapezoidal shape having essentially a uniform angular separation. Rounded corners 198a,198b between the gap and the periphery can be provided to avoid sharp edges which may damage tissue and to enhance comfort.

In this way the stopper can provide a penetration-resisting bearing surface extending beyond a maximum radial extent of the needle which can be dimensioned to bear directly or indirectly against at least part of the tissue surrounding an opening in said lacrimal gland during insertion of said implant into said lacrimal gland. In some embodiments the penetration-resisting bearing surface extends at least 0.1 mm beyond a maximum radial extent of said needle. For most applications this also means the penetration-resisting bearing surface extends beyond a maximum radial extent of the implant. In some embodiments the stopper can be substantially cylindrical and the penetration-resisting bearing surface can be substantially circular.

As shown in FIG. 21, when using an implant 153 having a flange 151 such as the implant shown in FIG. 12, the flange can be made of a flexible material such as silicone so that it folds within the axial bore of needle 152 when the implant is contained therein. Once the implant is ejected the flange resiliently springs back to its extended shape to contact the surrounding tissue as described above. This embodiment shows that the axial position of the stopper 156 can be adjustable 159 along the axis 154 of the needle in order to adjust the depth of the penetration of the needle which is especially important for proper placement of the flanged implant. The stopper can be formed as a separate washer-like body having a central hole 157 sized to form a friction fit over the outer surface 158 of the needle

Placement of the non-biodegradable drug-releasing implant can be as follows.

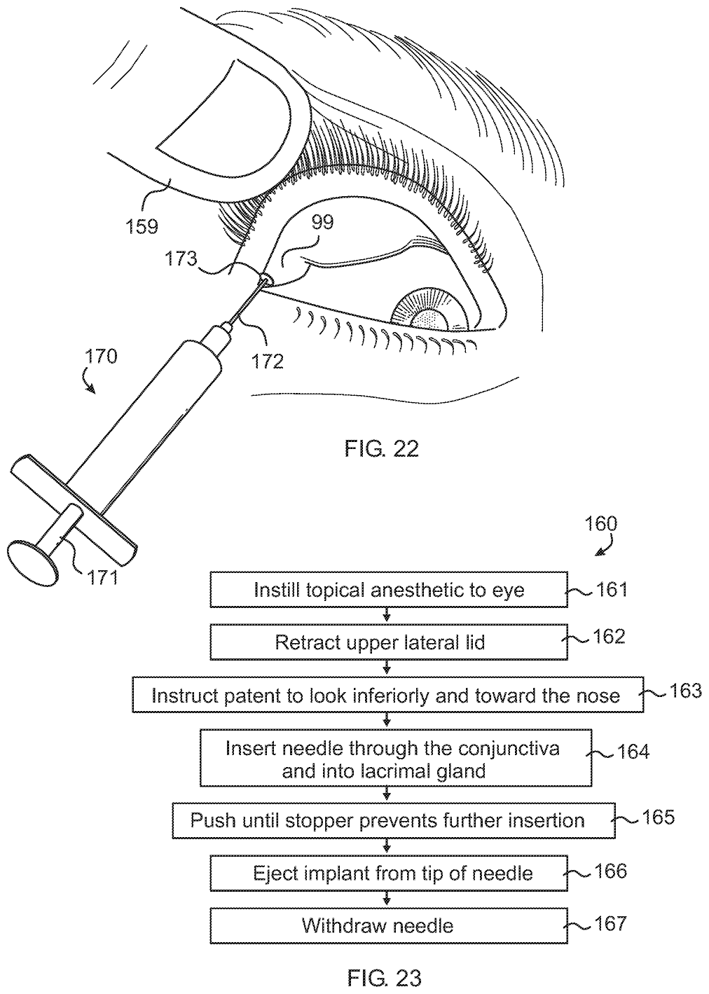

In reference to FIGS. 22-23, there is shown a method 160 for emplacing the non-biodegradable implant in the palpebral lobe of the lacrimal gland 99. The physician instills 161 a topical anesthetic on the surface of the eye. The physician then retracts 162 the lateral upper lid with his finger 159 or a retractor. The patient in instructed 163 to look inferiorly and toward the nose. The physician inserts 164 the needle 172 of the emplacement tool 170 through the conjunctiva into the lacrimal gland. The needle is pushed 165 into the lacrimal gland until the stopper 173 bears against the tissue surrounding the path of the needle and prevents the needle from going deeper. The plunger 171 is depressed to eject 166 the implant to the desired depth. The proximal flange of the implant will rest on the external surface of the lacrimal gland and in the case of the palpebral lobe on the conjunctiva. The remainder of the implant is within the lacrimal gland. The needle is then withdrawn 167. In some embodiments an adhesive surface or material is present on the surface of the flange that faces the lacrimal gland and conjunctiva. This helps prevent extrusion.

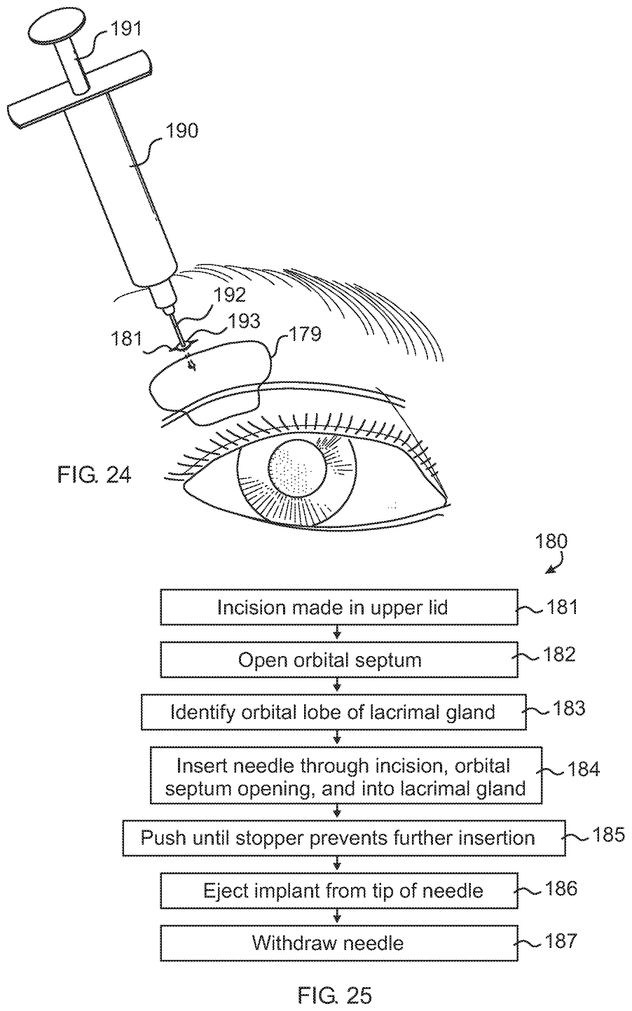

In reference to FIGS. 24-25, there is shown a method 180 for emplacing the non-biodegradable implant in the orbital lobe 179, a small incision 181 can be made in lateral upper lid. Blunt opening or sharp opening of orbital septum can be performed 182. The orbital lobe of the lacrimal gland is identified 183. The physician inserts the needle 192 through the incision, the orbital septum opening, and into the lacrimal gland 184. The tool 190 is pushed 185 distally into the lacrimal gland until the stopper 193 prevents it from going deeper. The plunger is depressed to eject 186 the implant to the desired depth. The proximal flange of the implant will rest on the external surface of the lacrimal gland. The remainder of the implant is within the lacrimal gland. The applicator is then removed 187. In some embodiments an adhesive surface or material is present on the surface of the flange that faces the conjunctiva. This helps prevent extrusion. The flange can also be sutured to the surface of the lacrimal gland.

Alternately, no incision is made. The tool needle can be inserted through the lateral upper lid, orbital septum and into the lacrimal gland. The stopper prevents the needle from going deeper than needed. The stopper on the tool is positioned so that the implant will be just beneath the skin and in the lacrimal gland. The plunger pushes the implant to the desired depth. The tool is then withdrawn.

In this way the present embodiments allow the entire ocular surface to be continuously coated with medication in a substantially uniform manner. The above-described embodiments can therefore allow maximum penetration of medication into the eye.

Dry Eye Syndrome

The drug releasing lacrimal gland implant may contain one or more drug releasing of a paraympathomimetic medication such as pilocarpine.

The drug releasing lacrimal gland implant may contain one or more of androgens such as testosterone, dihydrotestosterone, methyltestosterone, testosterone cypionate, oxandrolone, danazol or other androgens

The drug releasing implant may contain one or more of anti-infective medications. These can include ganciclovir, acyclovir, famcyclovir, gentamicin, tobramycin, moxifloxacin, levofloxacin, ocufloxacin, ciprofloxacin, sulfacetamide products, polymyxin, neomycin, penicillin, cephalosporins, doxycycline, tetracycline, minocycline, erythromycin, biaxin, trifluridine.

The drug releasing implant may contain one or more of anti-inflammatory medications. This may include dexamethasone, triamcinolone, fluocinolone, cyclosporine, lifitigrast.

The drug releasing implants may contain one or more of lubricants. The drug releasing implants may contain one or more of artificial tears or gels.

The drug-releasing implant to treat dry eye syndrome can be either biodegradable, bioabsorbable, non-biodegradable, or non-bioabsorbable, and can be emplaced using any of the above-described emplacement methods.

In this way the drug can be released by the implant over a designated period of time to diffuse into the lacrimal gland. This can allow maximal and direct treatment of the lacrimal gland without or with very minimal systemic or ocular side effects.

In this way the above embodiments allow the direct treatment of the lacrimal gland in addressing dry eye syndrome. Further the above embodiments can minimize systemic side effects of treatment by allowing local treatment of the lacrimal gland without the need for systemic therapy as for example with pilocarpine or androgens. In this way the above embodiments can prevent or minimize ocular side effects.

While the exemplary embodiments of the invention have been described, modifications can be made and other embodiments may be devised without departing from the spirit of the invention and the scope of the appended claims.

What is claimed is:

1. A method for delivering a drug to an eye or its associated lacrimal system to treat a condition, said method comprises:
placing an implant into a lacrimal gland of a patient, wherein the implant comprises:
a body comprising:
a drug-carrying region comprising:
a drug selected to treat said condition; and,
wherein said placing comprises:
positioning said implant so that an exposed surface of the drug-carrying region is directly exposed to glandular tissues; and,
diffusing of said drug into said glandular tissues.

2. The method of claim 1 which further comprises:
allowing secretions emanating from said glandular tissues to carry an amount of said drug diffused into said glandular tissues through one or more lacrimal gland secretory ducts, and onto the surface of the eye.

3. The method of claim 1 which further comprises:
selecting said drug to treat dry eye syndrome;
allowing said drug diffused into said glandular tissues to stimulate enhanced secretory activity; and,
increasing secretions emanating from said glandular tissues through a one or more lacrimal gland secretory ducts, and onto the surface of the eye.

4. The method of claim 1, wherein said implant consists of one or more biodegradable and/or bioabsorbable materials.

5. The method of claim 1, wherein said implant comprises one or more biodegradable and/or bioabsorbable materials.

6. The method of claim 1, wherein said implant comprises one or more non-biodegradable and/or non-bioabsorbable materials.

7. The method of claim 1, wherein said placing comprises inserting said implant in a palpebral lobe of said lacrimal gland.

8. The method of claim 1, wherein said placing comprises inserting said implant in an orbital lobe of said lacrimal gland.

9. The method of claim 1, wherein placing comprises:
inserting a first implant in an orbital lobe of said lacrimal gland; and
inserting a second implant in a palpebral lobe of said lacrimal gland.

10. The method of claim 1, wherein placing comprises:
inserting two or more of said implants into said lacrimal gland.

11. The method of claim 1, which further comprises:
removing said implant out of said lacrimal gland;
wherein said removing comprises:
grasping a part of said implant with a grasping tool.

12. The method of claim 1, which further comprises retaining said implant in the lacrimal gland during delivery of said drug onto said eye.

13. The method of claim 12, wherein said retaining occurs in absence of fixating said implant to the lacrimal gland with a clamp or other device apart from said implant.

14. The method of claim 12, wherein said retaining comprises providing said implant with at least one radially extended prominence.

15. The method of claim 12, wherein said retaining comprises providing said implant with at least one distal anchoring bulge.

16. The method of claim 12, wherein said retaining comprises providing said implant with a plurality of spaced apart anchoring bulges.

17. The method of claim 12, wherein said retaining comprises providing said implant with a proximal flange.

18. The method of claim 17, wherein said implant comprises an integrated combination of said body and said proximal flange; and wherein said drug-carrying region is imbedded in said lacrimal gland while said proximal flange is not.

19. The method of claim 17, wherein said proximal flange bears against the conjunctiva of said patient.

20. The method of claim 17, wherein said flange has a cross-sectional dimension greater than said drug-carrying region.

21. The method of claim 1, wherein said placing comprising inserting said implant through the conjunctiva of said patient.

22. The method of claim 1, wherein said placing comprising inserting said implant through an upper eyelid of said patient.

23. The method of claim 1, wherein said placing comprises:
instilling a topical anesthetic on the surface of the eye;
retracting the lateral upper eyelid;
instructing the patient to look inferiorly and toward the nose;
inserting a needle carrying said implant through the conjunctiva and into the lacrimal gland;
ejecting said implant out of said needle into the lacrimal gland; and,
withdrawing said needle from said conjunctiva.

24. The method of claim 23, wherein said inserting comprises:
pushing said needle until a stopper prevents further insertion;

25. The method of claim 1, wherein said placing comprises:
instilling a topical anesthetic on the surface of the eye;
forming an incision in the lateral upper eyelid;
creating an opening in the orbital septum;
instructing the patient to look inferiorly and toward the nose;
inserting a needle carrying said implant through the lateral upper eyelid, orbital septum and into the lacrimal gland;
ejecting said implant out of said needle into the lacrimal gland; and,
withdrawing said needle from said lateral upper eyelid.

26. The method of claim 25, wherein said inserting comprises:
pushing said needle until a stopper prevents further insertion.

27. A method for treating dry eye syndrome, said method comprises:
delivering an effective amount of a drug to the lacrimal gland of a patient;
wherein said delivering comprises:
placing an implant into a lacrimal gland of a patient, wherein the implant comprises:
a body comprising:
a drug-carrying region comprising:
a drug; and,
wherein said placing comprises:
positioning said implant so that an exposed surface of the drug-carrying region is directly exposed to glandular tissues; and,
diffusing of said drug into said glandular tissues.

28. An implant for delivering a drug to an eye or lacrimal system of a patient, said implant comprises:
a body comprising:
a drug-carrying region comprising:
a drug;
said implant being shaped and dimensioned to be inserted into the lacrimal gland of the patient;
whereby while the implant is inserted into the lacrimal gland of the patient a surface of said drug-carrying region is directly exposed to glandular tissues thereby allowing diffusion of said drug into said glandular tissues.

29. The implant of claim 28, wherein said body comprises a first biodegradable and/or bioabsorbable material.

30. The implant of claim 28, wherein said body comprises a first non-biodegradable and/or non-bioabsorbable material.

31. The implant of claim 28, which further comprises at least one radially extended prominence.

32. The implant of claim 31, wherein said prominence comprises at least one distal anchoring bulge.

33. The method of claim 31, wherein said radially extended prominence has a cross-sectional dimension greater than said drug-carrying region.

34. The implant of claim 28, which further comprises a plurality of spaced apart anchoring bulges extending radially from said body.

35. The implant of claim 28, which further comprises a proximal flange.

36. The implant of claim 35, wherein said drug carrying region is separated a distance from said proximal flange.

37. The implant of claim 35, wherein said drug carrying region and said flange are made from a unitary, integrated piece of material.

38. The implant of claim 35, wherein said proximal flange is made from a second biodegradable and/or bioabsorbable material.

39. The implant of claim 38, wherein said first biodegradable and/or bioabsorbable material and said second biodegradable and/or bioabsorbable material are identical.

40. The implant of claim 35, wherein said proximal flange comprises a distal flange surface oriented to rest against at least part of the tissue surrounding an opening in said lacrimal gland when said implant is properly emplaced in said lacrimal gland.

41. The implant of claim 29, wherein said first biodegradable and/or bioabsorbable material is selected from the group consisting of:
poly (lactic co glycolic acid) (PLGA);
hydroxymethylcellulose;
collagen;
polydioxanone;
E-Caprolactone-L-Lactide-copolymer;
polycaprolactone (PCL)/PLGA; and,
polyethylene glycol (PEG).

42. The implant of claim 28, wherein said body further comprises biodegradable particles selected from the group consisting of:
colloidal particles, liposomes, microparticles, nanoparticles and nanospheres

43. The implant of claim 28, wherein said drug is dispersed in biodegradable polymeric matrix or membrane.

44. The implant of claim 28, wherein said drug comprises a therapeutic agent selected from the group consisting of:
bimatoprost, latanoprost, tafluprost, travoprost, brinzolamide, betaxolol, carteolol, levobunolol, timolol, apraclonidine, brimonidine, ganciclovir, acyclovir, famcyclovir, gentamicin, tobramycin, moxifloxacin, levofloxacin, ocufloxacin, ciprofloxacin, sulfacetamide products, polymyxin, neomycin, penicillin, cephalosporins, doxycycline, tetracycline, minocycline, erythromycin, biaxin, trifluridine, dexamethasone, triamcinolone, fluocinolone, and cyclosporine.

45. The implant of claim 28, wherein said drug comprises a therapeutic agent selected to treat dry eye syndrome.

46. The implant of claim 28, wherein said body is coated with a biodegradable material.

47. The implant of claim 28, wherein said drug is conjugated to a biodegradable material.

48. The implant of claim 28, wherein implant establishes a flow of drug-containing fluid of at least 0.01 microliter per minute to said eye.

49. The combination of a drug-carrying implant and a tool for inserting said implant into the lacrimal gland;
wherein said implant comprises:
a drug-carrying region;
wherein said tool comprises:
a needle having an a sharpened distal tip, an axial bore extending to a distal aperture;
a ramrod shaped and dimensioned to slidingly engage said axial bore;
wherein said axial bore slidingly carries said implant therein.

51. The combination of claim 49, which further comprises a stopper radially extending from said needle separated an axial distance from said sharpened distal tip.

52. The combination of claim 51, wherein an axial position of said stopper is adjustable with respect to said distal tip.

53. The combination of claim 52, wherein said stopper provides a penetration-resisting bearing surface extending beyond a maximum radial extent of said needle.

54. The combination of claim 53, wherein said penetration-resisting bearing surface extends at least 0.1 mm beyond a maximum radial extent of said needle.

55. The combination of claim 53, wherein said penetration-resisting bearing surface extends beyond a maximum radial extent of said implant.

56. The combination of claim 55, wherein said penetration-resisting bearing surface extends at least 0.1 mm beyond a maximum radial extent of said implant.

57. The combination of claim 53, wherein said penetration-resisting bearing surface is dimensioned to bear directly or indirectly against at least part of the tissue surrounding an opening in said lacrimal gland during insertion of said implant into said lacrimal gland.

58. A tool for inserting a drug-releasing implant into the lacrimal gland of a patient, said tool comprises:
a hand-graspable member;
a needle extending distally from said member;
said needle having a distal segment elongated along an axis, said distal segment having a free distal end; and,
a stopper located an axial distance proximal from said distal end;
wherein said stopper comprises:
a radial prominence supporting a penetration-resisting bearing surface a radial distance from said distal segment;
wherein said penetration-resisting bearing surface is shaped, dimensioned, and located to directly or indirectly bear against at least part of the tissue surrounding an opening in said lacrimal gland during insertion of said implant into said gland.

59. The tool of claim 58, wherein said penetration-resisting bearing surface is shaped and dimensioned to prevent over-penetration of said distal segment into said gland.

60. A method for emplacing a drug-releasing implant into the lacrimal gland of a patient, wherein said implant has a proximal capping flange having a distal flange surface for resting against the tissue forming said gland, said method comprises:
selecting an implantation tool including a needle having a distal segment having a free distal end, and a stopper located an axial distance proximally from said distal end, said stopper supporting a penetration-resisting bearing surface having a radial dimension greater than a maximum radial dimension of said implant;
releasably carrying said implant within a bore of said needle;
puncturing said gland with said needle to form an opening in the surface of said gland;
pushing said needle axially into said lacrimal gland until said penetration-resisting bearing surface prevents further axial movement of said implantation tool;
releasing said implant from said implantation tool; and,
axially pulling said tool away from said implant.

61. The method of claim 60, wherein the penetration-resisting bearing surface directly contacts a portion of the tissue surrounding said opening thereby preventing over-insertion of the needle into the lacrimal gland.

62. The method of claim 60, wherein said selecting further comprises:
choosing an implantation tool so that said penetration-resisting bearing surface has a radial dimension which is at least 0.1 mm larger than the maximum radial dimension of the needle.
